(19) United States
(12) Patent Application Publication
(10) Pub. No.: US 2018/0171242 A1
(43) Pub. Date: Jun. 21, 2018

(54) EFFICIENT PROCESS FOR UPGRADING PARAFFINS TO GASOLINE

(71) Applicant: ExxonMobil Research and Engineering Company, Annandale, NJ (US)

(72) Inventor: Mohsen N. Harandi, New Hope, PA (US)

(21) Appl. No.: 15/825,206

(22) Filed: Nov. 29, 2017

Related U.S. Application Data
(60) Provisional application No. 62/434,488, filed on Dec. 15, 2016.

(51) Int. Cl. C10G 57/02; C10G 11/14; C10G 7/00; C10G 50/00; C10G 11/05; C07C 2/04

(52) U.S. Cl. CPC C10G 57/02; C10G 11/14; C10G 7/00; C10G 50/00; C10G 11/05; B01J 29/40; C10G 2400/02; C10G 2300/1085; C10G 2400/22; C10G 2400/20; C07C 2/04

(57) ABSTRACT

Methods and systems are disclosed for upgrading a paraffinic feed to gasoline. The system includes a fluidized cracker receiving a paraffin-containing feedstream and producing an olefin product therefrom, the olefin product comprising C4 olefins; a separation system receiving the olefinic product and separating an olefin-containing feed therefrom, the olefin-containing feed having an olefin content of at least about 10 wt %; and an oligomerization reaction system receiving the olefin-containing feed and exposing the olefin-containing feed to a conversion catalyst under first effective conversion conditions to form an oligomerized olefin effluent comprising C₅+ olefinic compounds.

### CROSS-REFERENCE TO RELATED APPLICATIONS

This application claims priority to U.S. Provisional Application Ser. No. 62/434,488 filed Dec. 15, 2016, which is herein incorporated by reference in its entirety.

### FIELD

This application relates to the field of upgrading paraffins to gasoline.

### BACKGROUND

Upgrading of paraffins, such as light paraffins, to more valuable products has been a focus of researchers for decades. In a common commercial example, fluidized cracking systems are used to convert a paraffin-containing feed to maximize the production of a particular olefin, usually ethylene. Such systems are generally operated at very high temperatures with steam and have expensive refrigeration and fractionation systems. It would be desirable to provide other more efficient pathways to convert paraffins to upgraded products.

### SUMMARY

In one aspect, a method is provided for upgrading a paraffinic feed. The method includes cracking and/or dehydrogenating a paraffin-containing feedstream in a cracking zone under conditions to produce an olefinic product stream; fractionating the olefinic product stream to obtain an olefin-containing feed; and exposing the olefin-containing feed having an olefin content of at least about 10 wt. % to a conversion catalyst under first effective conversion conditions to form an oligomerized olefin effluent comprising C5+ olefinic compounds, wherein the first effective conversion conditions comprise a pressure of less than about 300 psig (2.1 MPa) and a temperature of at least about 550° F. (288° C.).

In another aspect, a system is provided for upgrading a paraffinic feed. The system includes a fluidized cracker receiving a paraffin-containing feedstream and producing an olefin product therefrom, the olefin product comprising C4 olefins; a separation system receiving the olefinic product and separating an olefin-containing feed therefrom, the olefin-containing feed having an olefin content of at least about 10 wt %; and an oligomerization reaction system receiving the olefin-containing feed and exposing the olefin-containing feed to a conversion catalyst under first effective conversion conditions to form an oligomerized olefin effluent comprising C5+ olefinic compounds, wherein the first effective conversion conditions comprise a pressure of less than about 300 psig (2.1 MPa) and a temperature of at least about 550° F. (288° C.).

### DRAWINGS

FIG. 1 is a schematic illustrating an exemplary process of upgrading paraffins to gasoline according to one or more embodiments of the present invention.

### DETAILED DESCRIPTION

Systems and methods are provided for the efficient upgrading of paraffins to gasoline. In such systems and methods, improvements can be realized by using a common product recovery system for the effluent from the oligimerization reaction system and for a portion of hydrocarbon effluent from the fluidized cracker not fed to the oligimerization reaction system (e.g., wild naphtha). Further improvements can be realized by operating the fluidized cracker at lower pressures and temperatures, e.g., temperatures and pressures lower than that used to maximize ethylene production, which is often the case for fluidized cracking units. For example, the fluidized cracker may be operated under conditions to promote cracking and/or dehydrogenation of paraffins in the feed to maximize the production of C4 olefins and gasoline and distillate boiling range hydrocarbons.

In an aspect, a method is provided for upgrading a paraffinic feed comprising cracking and/or dehydrogenating a paraffin-containing feedstream in a cracking zone under conditions to produce an olefinic product stream. The olefinic product stream may include various olefins, including C4 olefins. The method may further include fractionating the olefinic product stream to obtain an olefin-containing feed, and exposing the olefin-containing feed having an olefin content of at least about 10 wt. % to a conversion catalyst under effective conversion conditions to form an oligomerized olefin effluent comprising C5+ olefinic compounds. The effective conversion conditions can include a pressure of less than about 300 psig, such as between 100 and 200 psig, and a temperature of at least about 550° F.

In an aspect, a system is provided for upgrading a paraffinic feed comprising a fluidized cracker receiving a paraffin-containing feedstream and producing an olefin product therefrom, the olefin product comprising C4 olefins. The system may further include a separation system receiving the olefinic product and separating an olefin-containing feed therefrom, the olefin-containing feed having an olefin content of at least about 10 wt %. The system may further include an oligomerization reaction system receiving the olefin-containing feed and exposing the olefin-containing geed to a conversion catalyst under first effective conversion conditions to form an oligomerized olefin effluent comprising C5+ olefinic compounds, wherein the first effective conversion conditions comprise a pressure of less than about 300 psig, such as between 100 psig to 200 psig, and a temperature of at least about 550° F.

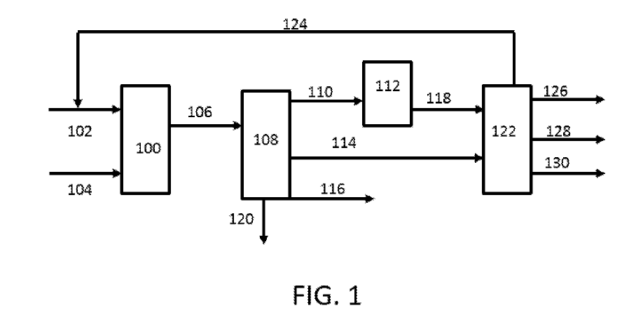

An exemplary embodiment of a system for implementing such a method is illustrated in FIG. 1. A paraffin containing feedstream 102 is fed to a fluidized cracking unit 100. In the illustrated embodiment, the fluidized cracking unit 100 is a steam cracker receiving steam 104 as a cofeed. Although the steam feed 104 is illustrated as being fed separately to the cracking unit 100 from paraffin-containing feed 102, it should be appreciated that the two streams 102, 104 may be contacted with each other prior to entering the cracking section of the cracking unit 100. Also, although not illustrated, the unit may have an auxiliary heating system for heating a catalyst that may be fed to the cracking unit 100 with the paraffin containing feed 102.

The cracking unit 100 may be operated under conditions to convert paraffins to olefins, preferably maximizing weight of C5+ and C2-C4 olefins, which may be efficently upgraded to gasoline boiling range hydrocarbons in a downstream oligomerization reaction system 112 as described further herein. The olefinic product stream 106 is first fed to a fractionation and compression system 108, where other product streams that are to bypass the oligomerization reaction system 112 can be separated, including a wild naphtha stream 114, corresponding to a naphtha boiling range fraction, and one or more distillate or heavier hydrocarbon product streams 116. In the case where the fluidized cracking unit 100 is a steam cracker, a water stream 120 may also be recovered.

The remaining olefin-containing fraction may be compressed to the oligomerization reaction system 112 pressure, such as between 100 psig and 200 psig, and fed to the oligomerization reaction system 112 as olefin containing feed 110. In the oligomerization reaction system 112, the olefin containing feed is exposed to a conversion catalyst under effective conversion conditions to form an oligomerized olefin effluent 118 comprising C5+ olefinic compounds as described in greater detail herein.

The oligomerized olefin effluent 118 and wild naphtha stream 114 are then fed to a common recovery system 122 in which various product streams are recovered, such as a hydrogen stream 126, a methane stream 128, and a gasoline boiling range product stream 130. Other light hydrocarbons including paraffins and light olefins can be recycled to the cracker 100 via recycle stream 124.

### Paraffin Containing Feedstream

Various paraffin containing feedstreams may be upgraded according to one or more embodiments of the present invention. In particular, the methods and systems disclosed herein may be advantageously employed with feedstreams containing light paraffins, such as C1-C5 alkanes. For example, the feed may be a liquified gas, such as liquified propane, butane, pentane or a mixture thereof. The feed may also be a crude that is a paraffin rich, or a fraction thereof, such as a vacuum gas oil. Other feed streams such as ethane, naphtha or distillate streams are also suitable feeds. Any mixture of the foregoing feeds or a mixture of any of the foregoing streams with another hydrocarbon stream is also contemplated.

### Fluidized Cracking

Fluidized cracking may be employed to convert paraffins in the paraffin-containing feedstream to olefins, such as through dehydrogenation and cracking. Advantageously, fluidized cracking may be performed at lower temperatures and/or pressures than that which is normally employed to maximize ethylene production. In aspects, the fludizied cracking can be operated under conditions to maximize the production of C4 olefins as well as gasoline and distillate boiling range molecules. For example, the fluidized cracking unit may be operated at pressures lower than 100 psi, such as lower than 40 psi. The fluidized cracking bed may be maintained at a temperature above 1200F, such as about 1350-1750 F. The per pass conversion of paraffins in the fluidized cracking unit may be more than 50 wt %, such as at least 75 wt % or at least 85 wt %. The residence time of the feed in the cracking zone of the reactor may be less than 20 seconds, preferably utilizing riser reactor.

The fluidized cracking may be performed with a catalyst, such as a temperature stable zeolite, such as Zeolite Y including FCC spent catalyst, a silicoaluminaphosphate (SAPO) catalyst, sand or silica, silica-alumina, or heated petroleum coke. The catlyst may further contain nickel and vanadium as a flexicoke or fulid coke may contain. Other dehdrogenation promoters such as Co or Mo are also acceptable. The catalyst material may be heated, such as in a regenerator system which also utlizes suplimental fuel buring to provide adequate heat of reaction for the cracking/dehydrogenation zone, before it is combined with the pafaraffin-containing feedstream and fed to the fluidized cracking unit.

The fluidized cracking may be performed with the presence of steam; however, in some embodiments fluidized cracking may be perfomed in the absence of steam or with minimal steam addition, e.g., less than 5%.

### Olefinic Product Stream

The paraffin-containing feedstream is dehydrogenated and/or cracked under conditions to produce an olefinic product stream. The olefinic product stream has a higher olefin content than the paraffin containing feedstream as a substantial portion of the paraffins contained in the feedstream, such as at least 50 wt %, are converted to olefins in the fluidized cracking unit. Furthermore, a substantial amount of the olefins may be C2-C4 olefins as well as C5+ olefins, such as C5-C9 olefins. The olefinc product stream may comprise less than 5 wt % ethylene.

### Fractionation of Olefinic Product Stream

Advantagously, the olefinic product stream may be fractionated and compressed to produce a compressed olefin containing feed. The compressed olefin containing feed may have an olefin content of at least 10 wt %, such as at least 25%, or at least 50%. The compressed olefin containing feed may comprise C2-C4 olefins as well as C5+ olefins, such as C5-C9 olefins.

Various other hydrocarbon fractions may be separated from the olefinic product stream to bypass the oligomerization reaction unit. For example, the fractionation and compression system may separate a wild naphtha stream, e.g., hydrocarbon fraction containing at least 90% of C7-C9 fraction from the olefinic product stream. The fractionation stream may also separate a distillate, e.g., hydrocarbon fraction boiling between 350° F. and 650° F., and/or heavier hydrocarbon product streams.

### Compressed Olefinic Feedstream

The olefin-containing feed can be any hydrocarbon feed that contains olefins. The olefin-containing feed may be compressed to the pressure of the downstream oligomerization reactor, e.g., between about 100 psi to about 200 psi. In some aspects, at least a portion of the olefin-containing feed can include one or more low value refinery streams, such as refinery fuel gas. In such aspects, the one or more low value streams may be present in the olefin-containing feed in an amount of at least about 10 wt. %, at least about 20 wt. %, at least about 30 wt. %, at least about 40 wt. %, at least about 50 wt. %, or at least about 60 wt. %. In the same or alternative aspects, the one or more low value streams may be present in the olefin-containing feed in an amount of about 100 wt. % or less, about 99 wt. % or less, about 95 wt. % or less, about 90 wt. % or less, about 80 wt. % or less, or about 70 wt. % or less.

In various aspects, the olefin-containing feed can include at least about 10 wt. % olefins, at least about 20 wt. %, at least about 30 wt. %, at least about 40 wt. %, at least about 50 wt. %, or at least about 60 wt. %. In the same or alternative aspects, the olefin-containing feed can include less than about 100 wt. % olefins, less than about 90 wt. %, less than about 80 wt. %, or less than about 70 wt. %.

In various aspects, the olefin-containing feed can include at least about 5 wt. % C1-C3 (or C1-C4) hydrocarbon compounds, with a portion being C2-C3 (or C2-C4) olefins, such as the olefin amounts listed above, at least about 10 wt. %, at least about 20 wt. %, at least about 30 wt. %, at least about 40 wt. %, at least about 50 wt. %, at least about 60 wt. %, or at least about 70 wt. %. In the same or alternative aspects, the olefin-containing feed can include less than about 100 wt. % C1-C3 (or C1-C4) hydrocarbon compounds, with a portion being C2-C3 (or C2-C4) olefins, such as the olefin amounts listed above, less than about 90 wt. %, less than about 80 wt. %, or less than about 70 wt. %. In certain aspects, the olefin-containing feed can include C1-C3 (or C1-C4) hydrocarbon compounds, with a portion being C2-C3 (or C2-C4) olefins, such that the C1-C3 (or C1-C4) hydrocarbon compounds are at least about 10 wt. % greater than the amount (wt. %) of C2-C3 (or C2-C4) olefins, at least about 20 wt. % greater, at least about 30 wt. % greater, at least about 40 wt. % greater, at least about 50 wt. % greater, or at least about 60 wt. % greater.

In various aspects, the olefin-containing feed can have a sulfur content of at least about 1 wppm, or at least about 100 wppm, or at least about 500 wppm, or at least about 1000 wppm, or at least about 1500 wppm. In another aspect, the sulfur content can be about 7000 wppm or less, or about 6000 wppm or less, or about 5000 wppm or less, or about 3000 wppm or less. The sulfur may be present as organically bound sulfur.

In one or more aspects, nitrogen can also be present in the olefin-containing feed. In an aspect, the amount of nitrogen can be at least about 1 wppm, or at least about 10 wppm, or at least about 20 wppm, or at least about 40 wppm. In another aspect, the nitrogen content can be about 250 wppm or less, or about 150 wppm or less, or about 100 wppm or less, or about 50 wppm or less.

It is appreciated that other olefin-containing feeds may be used in the processes disclosed herein and that the above-described feed properties are only exemplary.

### Oligomerization Reaction System

In various aspects, the olefin-containing feed can be exposed to an acidic catalyst (such as a zeolite) under effective conversion conditions for olefinic oligomerization and/or sulfur removal. Optionally, the zeolite or other acidic catalyst can also include a hydrogenation functionality, such as a Group VIII metal or other suitable metal that can activate hydrogenation/dehydrogenation reactions. The olefin-containing feed can be exposed to the acidic catalyst without providing substantial additional hydrogen to the reaction environment. Added hydrogen refers to hydrogen introduced as an input flow to the process, as opposed to any hydrogen that might be generated in-situ during processing. Exposing the feed to an acidic catalyst without providing substantial added hydrogen is defined herein as exposing the feed to the catalyst in the presence of a) less than about 100 SCF/bbl of added hydrogen, or less than about 50 SCF/bbl; b) a partial pressure of less than about 50 psig (350 kPag), or less than about 15 psig (100 kPag) of hydrogen; or c) a combination thereof. In an exemplary embodiment, no H2 is added to the oligomerization reaction zone.

The acidic catalyst used in the processes described herein can be a zeolite-based catalyst, that is, it can comprise an acidic zeolite in combination with a binder or matrix material such as alumina, silica, or silica-alumina, and optionally further in combination with a hydrogenation metal. More generally, the acidic catalyst can correspond to a molecular sieve (such as a zeolite) in combination with a binder, and optionally a hydrogenation metal. Molecular sieves for use in the catalysts can be medium pore size zeolites, such as those having the framework structure of ZSM-5, ZSM-11, ZSM-12, ZSM-22, ZSM-23, ZSM-35, ZSM-48, or MCM-22. Such molecular sieves can have a 10-member ring as the largest ring size in the framework structure. The medium pore size zeolites are a well-recognized class of zeolites and can be characterized as having a Constraint Index of 1 to 12. Constraint Index is determined as described in U.S. Pat. No. 4,016,218 incorporated herein by reference. Catalysts of this type are described in U.S. Pat. Nos. 4,827,069 and 4,992,067 which are incorporated herein by reference and to which reference is made for further details of such catalysts, zeolites and binder or matrix materials.

Additionally or alternately, catalysts based on large pore size framework structures (12-member rings) such as the synthetic faujasites, especially zeolite Y, such as in the form of zeolite USY. Zeolite beta may also be used as the zeolite component. Other materials of acidic functionality which may be used in the catalyst include the materials identified as MCM-36 and MCM-49. Still other materials can include other types of molecular sieves having suitable framework structures, such as silicoaluminophosphates (SAPOs), aluminosilicates having other heteroatoms in the framework structure, such as Ga, Sn, or Zn, or silicoaluminophosphates having other heteroatoms in the framework structure. Mordenite or other solid acid catalysts can also be used as the catalyst.

In various aspects, the exposure of the olefin-containing feed to the acidic catalyst can be performed in any convenient manner, such as exposing the olefin-containing feed to the acidic catalyst under fluidized bed conditions, moving bed conditions, and/or in a riser reactor. In some aspects, the particle size of the catalyst can be selected in accordance with the fluidization regime which is used in the process. Particle size distribution can be important for maintaining turbulent fluid bed conditions as described in U.S. Pat. No. 4,827,069 and incorporated herein by reference. Suitable particle sizes and distributions for operation of dense fluid bed and transport bed reaction zones are described in U.S. Pat. Nos. 4,827,069 and 4,992,607 both incorporated herein by reference. Particle sizes in both cases will normally be in the range of 10 to 300 microns, typically from 20 to 100 microns.

Acidic zeolite catalysts suitable for use as described herein can be those exhibiting high hydrogen transfer activity and having a zeolite structure of ZSM-5, ZSM-11, ZSM-12, ZSM-22, ZSM-23, ZSM-35, ZSM-48, MCM-22, MCM-36, MCM-49, zeolite Y, and zeolite beta. Such catalysts can be capable of oligomerizing olefins from the olefin-containing feed. For example, such catalysts can convert C2-C4 olefins, such as those present in a refinery fuel gas, to C5+ olefins. Such catalysts can also be capable of converting organic sulfur compounds such as mercaptans to hydrogen sulfide without added hydrogen by utilizing hydrogen present in the hydrocarbon feed. Group VIII metals such as nickel may be used as desulfurization promoters. A fluid-bed reactor/regenerator can assist with maintaining catalyst activity in comparison with a fixed-bed system. Further, the hydrogen sulfide produced in accordance with the processes described herein can be removed using conventional amine based absorption processes.

ZSM-5 crystalline structure is readily recognized by its X-ray diffraction pattern, which is described in U.S. Pat. No. 3,702,866. ZSM-11 is disclosed in U.S. Pat. No. 3,709,979, ZSM-12 is disclosed in U.S. Pat. No. 3,832,449, ZSM-22 is disclosed in U.S. Pat. No. 4,810,357, ZSM-23 is disclosed in U.S. Pat. Nos. 4,076,842 and 4,104,151, ZSM-35 is disclosed in U.S. Pat. No. 4,016,245, ZSM-48 is disclosed in U.S. Pat. No. 4,375,573 and MCM-22 is disclosed in U.S. Pat. No. 4,954,325. The U.S. Patents identified in this paragraph are incorporated herein by reference.

While suitable zeolites having a coordinated metal oxide to silica molar ratio of 20:1 to 200:1 or higher may be used, it can be advantageous to employ aluminosilicate ZSM-5 having a silica:alumina molar ratio of about 25:1 to 70:1, suitably modified. A typical zeolite catalyst component having Bronsted acid sites can comprises, consist essentially of, or consist of crystalline aluminosilicate having the structure of ZSM-5 zeolite with 5 to 95 wt. % silica, clay and/or alumina binder.

These siliceous zeolites can be employed in their acid forms, ion-exchanged or impregnated with one or more suitable metals, such as Ga, Pd, Zn, Ni, Co, Mo, P, and/or other metals of Periodic Groups III to VIII. The zeolite may include other components, generally one or more metals of group IB, IIB, IIIB, VA, VIA or VIIIA of the Periodic Table (IUPAC).

Useful hydrogenation components can include the noble metals of Group VIIIA, such as platinum, but other noble metals, such as palladium, gold, silver, rhenium or rhodium, may also be used. Base metal hydrogenation components may also be used, such as nickel, cobalt, molybdenum, tungsten, copper or zinc.

The catalyst materials may include two or more catalytic components which components may be present in admixture or combined in a unitary multifunctional solid particle.

In addition to the preferred aluminosilicates, the gallosilicate, ferrosilicate and “silicalite” materials may be employed. ZSM-5 zeolites can be useful in the process because of their regenerability, long life and stability under the extreme conditions of operation. Usually the zeolite crystals have a crystal size from about 0.01 to over 2 microns or more, such as 0.02-1 micron.

In various aspects, the catalyst particles can contain about 25 wt. % to about 40 wt. % H-ZSM-5 zeolite, based on total catalyst weight, contained within a silica-alumina matrix. Typical Alpha values for the catalyst can be about 100 or less. Sulfur conversion to hydrogen sulfide can increase as the alpha value increases.

The Alpha Test is described in U.S. Pat. No. 3,354,078, and in the Journal of Catalysis, Vol. 4, p. 527 (1965); Vol. 6, p. 278 (1966); and Vol. 61, p. 395 (1980), each incorporated herein by reference as to that description.

In various aspects, the olefin-containing feed may be exposed to the acidic catalyst by using a moving or fluid catalyst bed reactor. In such aspects, the catalyst may be regenerated, such via continuous oxidative regeneration. The extent of coke loading on the catalyst can then be continuously controlled by varying the severity and/or the frequency of regeneration. In a turbulent fluidized catalyst bed the conversion reactions are conducted in a vertical reactor column by passing hot reactant vapor upwardly through the reaction zone and/or reaction vessel at a velocity greater than dense bed transition velocity and less than transport velocity for the average catalyst particle. A continuous process is operated by withdrawing a portion of coked catalyst from the reaction zone and/or reaction vessel, oxidatively regenerating the withdrawn catalyst and returning regenerated catalyst to the reaction zone at a rate to control catalyst activity and reaction severity to affect feedstock conversion. Preferred fluid bed reactor systems are described in Avidan et al U.S. Pat. No. 4,547,616; Harandi & Owen U.S. Pat. No. 4,751,338; and in Tabak et al U.S. Pat. No. 4,579,999, incorporated herein by reference. In other aspects, other types of reactors can be used, such as fixed bed reactors, riser reactors, fluid bed reactors, and/or moving bed reactors.

In one or more aspects, effective conversion conditions for exposing the olefin-containing feed to an acidic catalyst can include a temperature of about 300° F. (149° C.) to about 900° F. (482° C.), or about 350° F. (177° C.) to about 850° F. (454° C.), or about 350° F. (177° C.) to about 800° F. (427° C.), or about 350° F. (177° C.) to about 750° F. (399° C.), or about 350° F. (177° C.) to about 700° F. (371° C.), or a temperature of at least about 400° F. (204° C.), or at least about 500° F. (260° C.), or at least about 550° F. (288° C.), or at least about 600° F. (316° C.); a pressure of about 50 psig (0.34 MPag) to about 1100 psig (7.6 MPag), or a pressure of about 100 psig (0.69 MPag) to about 1000 psig (6.9 MPag), or a pressure of about 100 psig (0.69 MPag) to about 200 psig (1.4 MPag), or about 150 psig (1.0 MPag) to about 975 psig (6.7 MPag), or about 200 psig (1.4 MPag) to about 950 psig (6.6 MPag), or about 250 psig (1.7 MPag) to about 900 psig (6.2 MPag), or about 300 psig (4.1 MPag) to about 850 psig (5.9 MPag), or about 300 psig (4.1 MPag) to about 800 psig (5.5 MPag), or a pressure of at least about 50 psig (0.34 MPag), or a pressure of at least about 100 psig (0.69 MPag), or a pressure of at least about 150 psig (1.0 MPag), or a pressure of at least about 200 psig (1.4 MPag), or a pressure of at least about 250 psig (1.7 MPag), or a pressure of at least about 300 psig (4.1 MPag), or a pressure of at least about 350 psig (2.4 MPag); and a total feed WHSV of about 0.05 hr−1 to about 40 hr−1, or about 0.05 to about 30 hr−1, or about 0.1 to about 20 hr−1, or about 0.1 to about 10 hr−1. Optionally, the total feed WHSV can be about 1 hr−1 to about 40 hr−1 to improve C5+ yield.

In addition to a total feed WHSV, a WHSV can also be specified for just the olefin compounds in the feed. In other words, an olefin WHSV represents a space velocity defined by just the weight of olefins in a feed relative to the weight of catalyst. In one or more aspects, the effective conversion conditions can include an olefin WHSV of at least about 0.1 hr−1, or at least about 1.0 hr−1, or at least about 2.0 hr−1, or at least about 3.0 hr−1, or at least about 4.0 hr−1, or at least about 5.0 hr−1, or at least about 8.0 hr−1, or at least about 10 hr−1, or at least about 15 hr−1. In the same or alternative aspects, the effective conversion conditions can include an olefin WHSV of about 40 hr−1 or less, or about 30 hr−1 or less, or about 20 hr−1 or less. In certain aspects, the effective conversion conditions can include an olefin WHSV of about 0.8 hr−1 to about 30 hr−1, or about 0.8 hr−1 to about 20 hr−1, or about 0.8 hr−1 to about 15 hr−1, or about 0.8 hr−1 to about 10 hr−1, or about 0.8 hr−1 to about 7 hr−1, or about 0.8 hr−1 to about 5 hr−1, or about 1.0 hr−1 to about 30 hr−1, or about 1.0 hr−1 to about 20 hr−1, or about 1.0 hr−1 to about 15 hr−1, or about 1.0 hr−1 to about 10 hr−1, or about 1.0 hr−1 to about 7 hr−1, or about 1.0 hr−1 to about 5 hr−1, or about 2.0 hr−1 to about 30 hr−1, or about 2.0 hr−1 to about 20 hr−1, or about 2.0 hr−1 to about 15 hr−1, or about 2.0 hr−1 to about 10 hr−1, or about 2.0 hr−1 to about 7 hr−1, or about 2.0 hr−1 to about 5 hr−1, about 4.0 hr−1 to about 30 hr−1, or about 4.0 hr−1 to about 20 hr−1, or about 4.0 hr−1 to about 15 hr−1, or about 4.0 hr−1 to about 10 hr−1, or about 4.0 hr−1 to about 7 hr−1. An olefin WHSV of about 1 hr−1 to about 40 hr−1 can be beneficial for increasing the C5+ yield.

In various aspects, decreasing the temperature below 800 F and increasing the olefin WHSV above 0.5 has positive impact on C5+ yield, e.g., when the olefin WHSV is increased above 1 hr−1, may improve product yield. For example, in such aspects, temperatures of about 600° F. (316° C.) to about 800° F. (427° C.), or about 650° F. (343° C.) to about 750° F. (399° C.) may aid in increasing product yield, such as the yield of C5+ compounds, when the olefin WHSV is increased above 1 hr−1.

### Oligomerized Effluent

In various aspects, exposing an olefin-containing feed to the conversion conditions discussed above can produce an oligomerized olefin effluent that includes naphtha boiling range compounds. In such aspects, the naphtha boiling range compounds in the oligomerized olefin effluent can include compounds with 5 or more carbon atoms (C5+ compounds) in an amount of at least about 70 wt. %, or at least about 75 wt. %. In one or more aspects, the naphtha boiling range compounds in the oligomerized effluent can include C5+ compounds in an amount of at least about 50 wt. % of the olefin-containing feed, at least about 55 wt. %, at least about 60 wt. %, at least about 65 wt. %, at least about 70 wt. %, or at least about 75 wt. %. In various aspects, the naphtha boiling range compounds in the oligomerized effluent can have an aromatic content of less than about 25 wt. %, less than about 15 wt. %, less than about 10 wt. %, or less than about 5 wt. %. In one or more aspects, the naphtha boiling range compounds in the oligomerized effluent can have a reduced sulfur content compared to the olefin-containing feed. In such aspects, the sulfur content of naphtha boiling range compounds in the oligomerized olefin effluent can be about 100 wppm or less, or about 75 wppm or less, or about 50 wppm or less, or about 30 wppm or less, or about 20 wppm or less, or about 10 wppm or less.

### Recovery Section

The oligomerized effluent and fractions separated from the olefinic product stream that bypassed the oligomerization reaction system may be recovered in a common recovery section. For example, the recovery section may receive streams of the oligomerized effluent and wild naphtha.

Various products may be separated and recovered in the recovery section including a gasoline boiling range product, e.g., a hydrocarbon fraction boiling between 100° F. and 427° F. Methane and hydrogen may also be separately recovered as products from the recovery section. Other light hydrocarbons, such as unconverted light paraffins, e.g., C2-C5 paraffins, and light olefins, e.g, C2-C4 olefins, may be recycled to the fluidized cracker up to extinction.

### EMBODIMENTS

The following embodiments are also considered:

### Embodiment 1

A method of upgrading a paraffinic feed comprising: cracking and/or dehydrogenating a paraffin-containing feedstream in a cracking zone under conditions to produce an olefinic product stream comprising at least C4 olefins; fractionating the olefinic product stream to obtain an olefin-containing feed; and exposing the olefin-containing feed having an olefin content of at least about 10 wt. % to a conversion catalyst under first effective conversion conditions to form an oligomerized olefin effluent comprising C5+ olefinic compounds, wherein the first effective conversion conditions comprise a pressure of less than about 300 psig (2.1 MPa) and a temperature of at least about 550° F. (288° C.).

### Embodiment 2

The method of any enumerated Embodiment, further comprising recovering a gasoline boiling range product from the oligomerized olefin effluent.

### Embodiment 3

The method of any enumerated Embodiment, further comprising obtaining a wild naphtha stream from the olefinic product stream.

### Embodiment 4

The method of any enumerated Embodiment, wherein the olefinic product stream comprises C2-C4 olefins.

### Embodiment 5

The method of any enumerated Embodiment, wherein the olefinic product stream comprises C2-C4 olefins and C5+ olefins, such as C5 to C9 olefins.

### Embodiment 6

The method of any enumerated Embodiment, the step of cracking the paraffin-containing feedstream includes contacting the feedstream with a heated media comprising petroleum coke.

### Embodiment 7

The method of any enumerated Embodiment, wherein the heated media further comprises nickel and vanadium.

### Embodiment 8

The method of any enumerated Embodiment, wherein the per pass conversion of paraffins in the cracking zone of at least 50%, at least 75%, at least 85%.

### Embodiment 9

The method of any enumerated Embodiment, wherein the olefin-containing feed has an olefin content of at least 50 wt %.

### Embodiment 10

The method of any enumerated Embodiment, wherein the temperature of the first effective conversion conditions is less than about 800° F. (427° C.).

### Embodiment 11

A system for upgrading a paraffinic feed comprising: a fluidized cracker receiving a paraffin-containing feedstream and producing an olefin product therefrom, the olefin product comprising C4 olefins; a separation system receiving the olefinic product and separating an olefin-containing feed therefrom, the olefin-containing feed having an olefin content of at least about 10 wt %; and an oligomerization reaction system receiving the olefin-containing feed and exposing the olefin-containing feed to a conversion catalyst under first effective conversion conditions to form an oligomerized olefin effluent comprising C5+ olefinic compounds, wherein the first effective conversion conditions comprise a pressure of less than about 300 psig (2.1 MPa) and a temperature of at least about 550° F. (288° C.).

### Embodiment 12

The system of any enumerated Embodiment, further comprising a recovery section recovering a gasoline boiling range product from the oligomerized olefin effluent.

### Embodiment 13

The system of any enumerated Embodiment, wherein the separation system further separates a wild naphtha stream from the olefinic product.

### Embodiment 14

The system of any enumerated Embodiment, wherein the olefinic product comprises C2-C4 olefins.

### Embodiment 15

The system of any enumerated Embodiment, wherein the olefinic product comprises C2-C4 olefins and C5+ olefins, such as C5 to C9 olefins.

### Embodiment 16

The system of any enumerated Embodiment, wherein the fluidized cracker exposes the paraffin-containing feedstream with a heated media comprising petroleum coke.

### Embodiment 17

The system of any enumerated Embodiment, wherein the heated media further comprises nickel and vanadium.

### Embodiment 18

The system of any enumerated Embodiment, wherein the per pass conversion of paraffins in the cracking zone of at least 50%, at least 75%, at least 85%.

### Embodiment 19

The system of any enumerated Embodiment, wherein the olefin-containing feed has an olefin content of at least 50 wt %.

### Embodiment 20

The system of any enumerated Embodiment, wherein the temperature of the first effective conversion conditions is less than about 800° F. (427° C.).

1. A method of upgrading a paraffinic feed comprising:
cracking and/or dehydrogenating a paraffin-containing feedstream in a cracking zone under conditions to produce an olefinic product stream comprising at least C4 olefins;
fractionating the olefinic product stream to obtain an olefin-containing feed; and
exposing the olefin-containing feed having an olefin content of at least about 10 wt. % to a conversion catalyst under first effective conversion conditions to form an oligomerized olefin effluent comprising C5+ olefinic compounds, wherein the first effective conversion conditions comprise a pressure of less than about 300 psig (2.1 MPa) and a temperature of at least about 550° F. (288° C.).

2. The method of claim 1, further comprising recovering a gasoline boiling range product from the oligomerized olefin effluent.

3. The method of claim 1, further comprising obtaining a wild naphtha stream from the olefinic product stream.

4. The method of claim 1, wherein the olefinic product stream comprises C2-C4 olefins.

5. The method of claim 1, wherein the olefinic product stream comprises C2-C4 olefins and C5+ olefins, such as C5 to C9 olefins.

6. The method of claim 1, the step of cracking the paraffin-containing feedstream includes contacting the feedstream with a heated media comprising petroleum coke.

7. The method of claim 6, wherein the heated media further comprises nickel and vanadium.

8. The method of claim 1, wherein the per pass conversion of paraffins in the cracking zone of at least 50%, at least 75%, at least 85%.

9. The method of claim 1, wherein the olefin-containing feed has an olefin content of at least 50 wt %.

10. The method of claim 1, wherein the temperature of the first effective conversion conditions is less than about 800° F. (427° C.).

11. A system for upgrading a paraffinic feed comprising:
a fluidized cracker receiving a paraffin-containing feedstream and producing an olefin product therefrom, the olefin product comprising C4 olefins;
a separation system receiving the olefinic product and separating an olefin-containing feed therefrom, the olefin-containing feed having an olefin content of at least about 10 wt %; and
an oligomerization reaction system receiving the olefin-containing feed and exposing the olefin-containing feed to a conversion catalyst under first effective conversion conditions to form an oligomerized olefin effluent comprising C5+ olefinic compounds, wherein the first effective conversion conditions comprise a pressure of less than about 300 psig (2.1 MPa) and a temperature of at least about 550° F. (288° C.).

12. The system of claim 11, further comprising a recovery section recovering a gasoline boiling range product from the oligomerized olefin effluent.

13. The system of claim 11, wherein the separation system further separates a wild naphtha stream from the olefinic product.

14. The system of claim 11, wherein the olefinic product comprises C2-C4 olefins.

15. The system of claim 11, wherein the olefinic product comprises C2-C4 olefins and C5+ olefins, such as C5 to C9 olefins.

16. The system of claim 11, wherein the fluidized cracker exposes the paraffin-containing feedstream with a heated media comprising petroleum coke.

17. The system of claim 16, wherein the heated media further comprises nickel and vanadium.

18. The system of claim 11, wherein the per pass conversion of paraffins in the cracking zone of at least 50%, at least 75%, at least 85%.

19. The system of claim 11, wherein the olefin-containing feed has an olefin content of at least 50 wt %.

20. The system of claim 11, wherein the temperature of the first effective conversion conditions is less than about 800° F. (427° C.).
